(19) United States
(12) Patent Application Publication
(10) Pub. No.: US 2025/0017722 A1
(43) Pub. Date: Jan. 16, 2025

(54) METHOD FOR OPTIMISING THE FORMULAE FOR CALCULATING IMPLANT POWER

(71) Applicant: FONDATION A DE ROTHSCHILD, PARIS CEDEX 19 (FR)

(72) Inventors: Damien GATINEL, Paris (FR); Guillaume DEBELLEMANIÈRE, PIRAE, Tahiti Moorea (PF)

(21) Appl. No.: 18/714,270

(22) PCT Filed: Nov. 24, 2022
(86) PCT No.: PCT/FR2022/052174

§ 371 Date: May 29, 2024

(30) Foreign Application Priority Data
Nov. 29, 2021 (FR) FR2112685

(51) Int. Cl. A61F 2/16

(52) U.S. Cl. CPC A61F 2/16

(57) ABSTRACT

This process characterises a target system comprising a target implant and a target eye, said target system being characterised by an optical performance (CP) and a power (P) of said target implant. It comprises the following steps:
obtaining (E10) an adjustment value of optical property (C1) intended to adjust at least one adjustable optical property (PA) of said target system (SC),
obtaining (E20) an adjustment value of optical performance (C2) of said target system;
determining (E30, E30′) a correspondence between said power (P) and the optical performance (CP) from at least one optical property (PO) of the target system including at least one adjustable property (PA), of said adjustment value of optical property (C1) and of said performance adjustment value (C2), the determining (E30) comprising the steps of:
   (i) adjustment (E30A) of a value of said adjustable property (PA) with said adjustment value of optical property (C1),
   (ii) adjustment (E30B) of a value of said optical performance (CP) or of said power (P) with said performance adjustment value (C2).

### BACKGROUND OF THE INVENTION

The invention relates to the field of ocular implants.

Cataract surgery consists of replacing the crystalline lens by an artificial implant. The properties of this artificial implant must be determined prior to the operation to satisfy a refractive correction lens of the eye being operated on. This characterisation of the implant requires determining its power, expressed in diopters.

This determining comprises in particular a step for prediction of a postoperative property of the eye and of the implant, for example the expected position of the implant after it is stabilised in the eye.

A predicted power calculated on the basis of such a poorly estimated postoperative property can cause effective correction of the eye operated on relatively far from the target of refractive correction.

The difference between the effective refractive correction after operation and the preferred refractive correction prior to operation will be called “performance error”.

In the publication https://www.karger.com/Article/Pdf/514916 it has been proposed to optimise prediction of the power of an implant by adjusting the predicted position of this implant by taking into consideration adjustment constants in calculating this position so as to cancel a bias in the prediction of the refractive correction or minimise an error on this same prediction, for example the average quadratic error or the average absolute error.

The prior art comprises the application EP 2605696 A1.

However, the characterisation of an implant with this method of optimisation is unsatisfactory as it does not allow for predicting reliable values of refractive correction.

The aim of the invention especially is to eliminate these disadvantages.

### OBJECT AND SUMMARY OF THE INVENTION

Therefore, and according to a first aspect, the invention relates to a process executed by computer for characterising a target system comprising a target implant and a target eye, said target system being characterised by an optical performance (CP) and a power (P) of said target implant, said process comprising the following steps:
obtaining an adjustment value of optical property intended to adjust at least one adjustable optical property of said target system,
obtaining an adjustment value of optical performance of said target system;
determining a correspondence between said power and the optical performance from at least one optical property of the target system including at least one adjustable property, of said adjustment value of optical property and of said performance adjustment value, at least one of said optical properties being obtained from measurements (for example biometrics) of the target eye;
said determining of a correspondence between said power and the optical performance being:
   determining of said optical performance as a function of said power; or
   determining of said power (P) as a function of said optical performance, the determining comprising the steps of:
      (i) adjustment of a value of said adjustable property with said adjustment value of optical property,
      (ii) adjustment of a value of said optical performance or of said power with said performance adjustment value,
said adjustment value of optical property having been determined to minimise dispersion of errors, each of said errors being:
   (i) a difference between an initially predicted value, from a given value of said power, of said optical performance and a measured value of said optical performance, or
   (ii) a difference between an initially predicted value, from the measured value of said optical performance, of said power and the given value of said power, for a reference system of a type of said target system and comprising a reference eye and a reference implant;
said adjustment value of a performance having been determined so as to cancel a bias of said errors obtained when said dispersion is minimal.

Correlatively, the invention also proposes a device for characterisation of a target system comprising a target implant and a target eye, said target system being characterised by an optical performance and a power of said target implant, said process comprising:
a module for obtaining an adjustment value of optical property, intended to adjust at least one adjustable optical property of said target system,
a module for obtaining an adjustment value of optical performance of said target system;
a module for determining a correspondence between said power and the optical performance from at least one optical property of the target system including at least one adjustable property, of said adjustment value of optical property and of said performance adjustment value, at least one of said optical properties being obtained from measurements (for example biometrics) of the target eye;
the determining of a correspondence between said power and the optical performance being:
   determining of said optical performance as a function of said power; or
   determining of said power as a function of said optical performance, the determining comprising the steps of:
      (i) adjustment of a value of said adjustable property with said adjustment value of optical property,
      (ii) adjustment of a value of said optical performance or of said power with said performance adjustment value,
said adjustment value of optical property having been determined to minimise dispersion (SDA) of errors, each of said errors being:
   (i) a difference between an initially predicted value, from a given value of said power (P), of said optical performance and a measured value of said optical performance, or
   (ii) a difference between an initially predicted value, from the measured value of said optical performance, of said power and the given value of said power,
for a reference system of a type of said target system and comprising a reference eye and a reference implant;
said performance adjustment value having been determined so as to cancel a bias of said errors obtained when said dispersion is minimal.

In this document, “eye implant system” refers to a set comprising at least one eye and an implant, and optionally other optical elements, for example spectacles or lenses.

In this document, and for the sake of simplification:
“optical property” will refer to the property as such (for example the curvature of the cornea) or a value of this property (for example a curvature of 45 diopters); and
“performance” will refer to the performance as such (for example a refractive correction) or a value of this performance (for example a refractive correction of 2 diopters).

It should be specified that refractive correction makes reference to correction of the vision of the eye by the implant, equivalent to the correction which would be produced by a lens, spectacles or another device placed at a given distance from the eye.

In this way, and in general, for a given type of eye implant system and in a first determining phase, the invention proposes determining two adjustment values from a set of eye implant reference systems of the same type whereof the optical performances measured after operation are known, and whereof the powers of the implants are also known.

These first and second adjustment values are determined in such a way that:
(i) when the predicted optical performances or the predicted implant powers of each of the reference systems are adjusted by this same first adjustment value, or more precisely when the adjustment value is taken into account in the predicted position of each of the reference implants, the dispersion of errors between the optical performances or the predicted and known powers is minimised;
(ii) when the optical performances or the predicted implant powers of each of the reference systems are adjusted by this same second adjustment value, the bias of errors between the optical performances or the predicted and known powers is cancelled.

In this way, the two criteria (dispersion and bias) characterising the reliability of the predictions for the reference systems are jointly minimised by the two adjustment values being taken into account in the calculation of the optical performance or of the power.

In a second characterisation phase, when it comes to determining the optical performance of a target system of the same type or power of a target implant, these two adjustment values are used.

The act of cancelling the bias produces accurate predictions (“accurate” in English).

But at the same time the act of minimising dispersion and cancelling bias produces predictions not only accurate but also precise (“precise” in English), which is not a factor of the methods of the prior art, including application EP 2605696 A1.

The invention proposes using two adjustment values to achieve this aim.

In particular, taking into account the first adjustment value minimising dispersion diminishes the uncertainty over predictions. In fact, the dispersion of prediction errors over a set of reference systems quantifies the spread between the smallest and the biggest of these errors. If this spread is low (i.e. if the dispersion is minor), the predictions are said to be precise.

It might be noted here that precise predictions (“precise” in English) are not necessarily accurate (“accurate” in English). Inversely, accurate predictions are not perforce precise. Predictions are qualified as accurate if the average error (also called bias) on these predictions is close to zero. The skilled person sometimes uses the term “biased” to qualify predictions with low accuracy, and “non-biased” to qualify accurate predictions.

The methods of the prior art, even though they minimise error criterion, for example the average quadratic error or the average bias, fail to ensure that the dispersion of errors is minimal. They seek only to attain good predictions on average. But these methods can lead to significant prediction errors for some samples.

The inventors have observed that the fact of using a single adjustment value to cancel bias or minimise prediction errors of optical performance or implant power, as in the prior art, does not minimise dispersion of these errors.

In addition, and highly advantageously, in the first phase and in the first instance actually determining the first value which minimises dispersion, then in the second instance the second value which cancels the bias is simple to carry out, and in any case much simpler than a method which would aim to adjust several parameters simultaneously.

Determining the optical performance of the eye implant system or the power of the implant of this system prior to surgery for placing of the implant requires at least one optical property characterising this system after surgery. In an embodiment, the effective implant-cornea distance is used. This distance does not correspond to the implant-cornea distance observed immediately after placing of the implant, since the implant undergoes minor shifts during the days following surgery, before stabilising.

The prediction process according to the invention comprises obtaining an adjustable optical property. This can be subject to error, for example if it has been predicted or measured using an inaccurate method or equipment. This is the case in particular of the effective implant-cornea distance.

It is considered that this error dominates the rest of the errors in the predictions. This is generally due to the fact that the error caused by the inaccuracies in the measurements of other optical properties, for example those deriving from biometric measurements, is negligible relative to the error on the adjustable property. It is therefore judicious to select an adjustable optical property subject to significant prediction or measuring errors.

The adjustable optical property can be obtained by any means.

In any case, only the value of the adjustable optical property must be known. When the adjustable optical property is predicted, its method for obtaining can remain secret.

The characterisation process utilises a second adjustment value, said performance value, to cancel the bias of the predictions.

This performance adjustment value can typically be subtracted from the optical performance or from the initially predicted power to remove the bias on this initially predicted performance or power prediction.

The characterisation process sets up a correspondence between the power of the target implant and the optical performance of the eye implant target system. According to the implementation of the invention:
either the power of the target implant is given, and the characterisation process deducts the optical performance of the system from it;
or the optical performance of the target power system is given, and the characterisation process deducts the power of the target implant from it.

In particular, the characterisation process utilises measurements (to obtain optical properties) such as input data and forms part of an indirect measuring method which calculates or predicts a physical property (implant power or an optical performance) of an existing real object (an implant in an eye implant system).

According to a second aspect, the invention comprises a process executed by computer for determining adjustment values intended to characterise a target system comprising a target implant and a target eye, said target system being characterised by an optical performance and a power of said target implant, said process comprising the following steps:
obtaining a history comprising, for at least one reference system of a type of said target system, a datum comprising:
   (i) a measured value of its optical performance,
   (ii) at least one of its optical properties including at least one adjustable property; and
   (iii) a given value of the power of its reference implant;
calculation as a function of the adjustment variable for all the reference systems:
   (i) for said at least one reference system, and by using its actual datum, of an error being:
      (i-a) a difference between an initially predicted value and said measured value of its optical performance, or
      (i-b) a difference between an initially predicted value and said given value of the power of its implant;
   (ii) of dispersion of said errors obtained for a set of said reference systems;
determining of an adjustment value of optical property intended to adjust at least one adjustable property of said target system and corresponding to the value of said adjustment variable which minimises said dispersion, and
determining a performance adjustment value, equal to an average of said errors when said adjustment variable is equal to said adjustment value of optical property, and intended to adjust said optical performance or said power of the target system.

Correlatively, the invention also proposes a device for determining adjustment values intended to characterise a target system comprising a target implant and a target eye, said target system being characterised by an optical performance and a power of said target implant, said process comprising:
a module for obtaining a history comprising, for at least one reference system of a type of said target system, a datum comprising:
   (i) a measured value of its optical performance,
   (ii) at least one of its optical properties including at least one adjustable property; and
   (iii) a given value of the power of its reference implant;
a calculation module as a function of the same adjustment variable for all reference systems:
   (i) for said at least one reference system, and by using its actual datum, of an error being:
      (i-a) a difference between an initially predicted value and said measured value of its optical performance, or
      (i-b) a difference between an initially predicted value and said given value of the power of its implant;
   (ii) of dispersion of said errors obtained for a set of said reference systems;
a module for determining an adjustment value of optical property intended to adjust at least one adjustable property of said target system and corresponding to the value of said adjustment variable which minimises said dispersion, and
a module for determining a performance adjustment value, equal to an average of said errors when said adjustment variable is equal to said adjustment value of optical property, and intended to adjust said optical performance or said power of the target system.

In general, from a history of data corresponding for example to a series of pre- and postoperative results of surgery for placing implants, this process determines the adjustment values which, based on carrying out a characterisation process such as mentioned hereinabove, will serve to characterise an eye implant system.

By way of example, in these two aspects the invention can be utilised by a surgeon who, during his operations concerning a type of eye implant system, progressively builds a history comprising for each postoperative eye implant system of this type optical properties of this system including at least one adjustable optical property, the power of the implant and a measured value of an optical performance of this system, for example a refractive correction.

This history enables him to obtain the two adjustment variables for eye implant systems of this type.

In this example, when the surgeon contemplates operating on the eye of a new patient, he envisages an implant to be placed in this patient and accordingly determines a type of eye implant system.

Still in this example, using biometric measurements for example the surgeon obtains the optical properties of the eye implant system needed to characterise the eye implant system of the type determined. He utilises the two adjustment variables previously obtained to characterise the envisaged eye implant system. In this way for example, the surgeon achieves an implant power necessary for achieving a certain refractive correction after stabilisation of the implant in the eye.

In keeping with the invention, a “type of eye implant system” denotes any set of characteristics specific to an element of the system (type of eye, age of eye, type of implant, optical design or material of the implant, stabilisation system of the implant) or specific to interaction between these elements (type of installation of the implant in the eye).

In this way, the characterisation process utilises two adjustment values determined by the determining process for a type of eye implant system.

Two implants of the same power but of different types (for example of different materials) can provide a different refractive correction (or any other optical performance) in the same eye. It is therefore advantageous to consider the type of eye implant system in processes for determining and characterising as presented hereinabove.

Also, determining a minimum of dispersion can therefore be done in different ways.

According to a particular embodiment of the determining process, said adjustment variable which minimises said dispersion is determined by a gradient descent algorithm of said dispersion according to said variable, or by an algorithm consisting of calculating said dispersion for a plurality of values of said adjustment variable, and selecting the value which minimises said dispersion.

The choice of method can be motivated by different restrictions to precision and/or time.

According to a particular embodiment of the characterisation process, the adjustable optical property is a distance between the target implant and one of the elements of the target eye.

In the same way, according to a particular embodiment of the determining process, the adjustable optical property of a reference system corresponds to a distance between the reference implant and one of the elements of the reference eye.

According to a particular embodiment of the characterisation process, at least one of the optical properties of the target system is obtained from biometric measurements of the target eye, for example with a biometer.

In the same way, according to a particular embodiment of the determining process at least one of the optical properties of a reference system is obtained from biometric measurements of the target eye of this reference system.

According to an embodiment of the invention, at least one of the optical adjustable properties of a reference system or of a target system is predicted or measured.

According to an embodiment of the characterisation process, at least one of the adjustable optical properties is calculated from at least one optical property of the eye implant target system. This is especially the case of the effective implant-cornea distance which can be calculated from optical properties of the eye (such as the length of the eye, the curvature of the cornea, the width of the cornea etc.) and optionally of the implant (such as its thickness, its curvature, etc.).

In the same way, according to an embodiment of the determining process, at least one of the adjustable optical properties of at least one reference system is calculated from at least one of said optical properties of this reference system, for example of the eye.

The formulae enabling these calculations can be determined using various methods.

In a particular embodiment of the characterisation process, the calculation of at least one adjustable optical property is determined from a statistical regression or machine learning technique.

For example, if there are properties of multiple eyes and for each of these eyes there is a measured value of an adjustable property, for example the effective implant-cornea distance measured after placing of the implant, a method of regression or machine learning can be used to construct a formula for obtaining predictions of this adjustable property (from the properties of each eye) which are the closest possible to the measured values of this adjustable property.

In the same way, according to a particular embodiment of the determining process the calculation of at least one of said adjustable properties of at least one reference system is determined from a regression or machine learning technique.

According to a particular embodiment of the characterisation process, the determining of said adjustment value of optical property and of said performance adjustment value is carried out by a determining process according to one of the embodiments of the determining process described previously.

According to a particular embodiment of one of the processes such as described previously, the optical performance corresponds to a property from:
a refraction,
a refractive correction,
a position for focusing refracted light rays,
a correction of contrast.

The implant can act to produce a convergence of light rays onto a precise point of the eye, for example the retina. This convergence objective can be expressed by a position relative to the cornea or any other element of the eye, a refraction index, or an optical power expressed in diopters.

The implant can also be intended to improve the contrast of the retinal image for a spatial frequency or a group of given spatial frequencies.

In an embodiment, a single adjustment value of optical property and a single performance adjustment value are used.

This embodiment with a reduced number of adjustable parameters advantageously improves the capacity of the model to make predictions on novel data with a quality close to predictions on data utilised to construct this model.

The invention also proposes a system comprising at least one computer unit, for example a biometer, comprising at least one characterisation device and/or a device for determining such as described previously.

In this way, the invention applies several configurations, especially:
a characterisation device only,
a determining device only, or
a system combining both devices.

In an embodiment of the invention, the device for determining adjustment values is a computer which can communicate these values to a biometer comprising a characterisation device.

In another embodiment of the invention, the characterisation device is integrated into a biometer. Such a biometer can especially obtain optical properties by biometric measurements and utilise these optical properties to characterise an eye implant system.

It is also possible to harvest biometric data provided by a third party. In this way, a computer can be used to carry out the characterisation process and the determining process, by dispensing with a biometer.

The invention proposes a computer program comprising instructions for executing the steps of a characterisation process according to any one of the embodiments described previously.

The invention proposes a computer program comprising instructions for executing the steps of a determining process according to any one of the embodiments described previously.

It should be noted that the computer programs mentioned in the present presentation can utilise any programming language, and be in the form of source code, object code, or of intermediate code between source code and object code, such as in a partially compiled form, or in any other preferred form.

The invention also proposes a storage medium readable by computer equipment and/or a biometer of a computer program comprising instructions for executing the steps of a characterisation process according to one of the modes described previously.

The invention also proposes a storage medium readable by computer equipment and/or a biometer, a computer program comprising instructions for executing the steps of a determining process according to one of the modes described previously.

The recording media mentioned in the present exposé can be any entity or device capable of storing the program and being read by a biometer or by any computer equipment, especially a computer.

For example, the medium can comprise storage means, or even magnetic recording means, for example a hard drive.

Alternatively, the recording media can correspond to a circuit integrated into a computer or a biometer, a circuit into which the program is incorporated and adapted to execute a process such as described previously or to be used in executing this process.

### BRIEF DESCRIPTION OF THE FIGURES

Other characteristics and advantages of the present invention will emerge from the description given hereinbelow, in reference to the attached drawings which illustrate embodiments devoid of any limiting character.

FIG. 1 illustrates a process for determining adjustment values in keeping with a particular embodiment of the invention.

FIG. 2 illustrates a datum of a history which can be used in a particular embodiment of the invention.

FIG. 3 illustrates adjustment values in terms of the invention.

FIG. 4 illustrates an example of dispersion, of a bias and an average absolute error as a function of an adjustment variable of an adjustable property according to a particular embodiment of the invention.

FIG. 5 illustrates a device for determining of adjustment values in keeping with a particular embodiment of the invention.

FIG. 6 illustrates two embodiments of a characterisation process of a target system in keeping with a particular embodiment of the invention.

FIG. 7 illustrates a characterisation device of a target system in keeping with a particular embodiment of the invention.

FIG. 8 illustrates a first system according to a particular embodiment of the invention.

FIG. 9 illustrates a second system according to a particular embodiment of the invention.

FIG. 10 illustrates a third system according to a particular embodiment of the invention.

FIG. 11 illustrates a fourth system according to a particular embodiment of the invention.

FIG. 12 illustrates the hardware architecture of a device for determining according to a particular embodiment of the invention.

FIG. 13 illustrates the hardware architecture of a characterisation device according to a particular embodiment of the invention.

### Description of Particular Embodiments of the Invention

Several embodiments of the invention will now be described. In general, and as mentioned previously, the invention proposes a process for determining adjustment values which can be used to characterise a target system, and a characterisation process of a target system using such adjustment values.

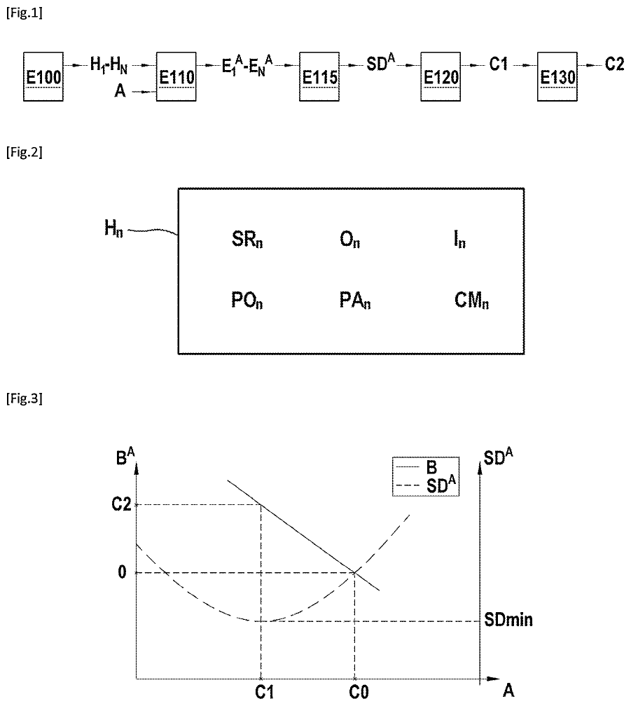

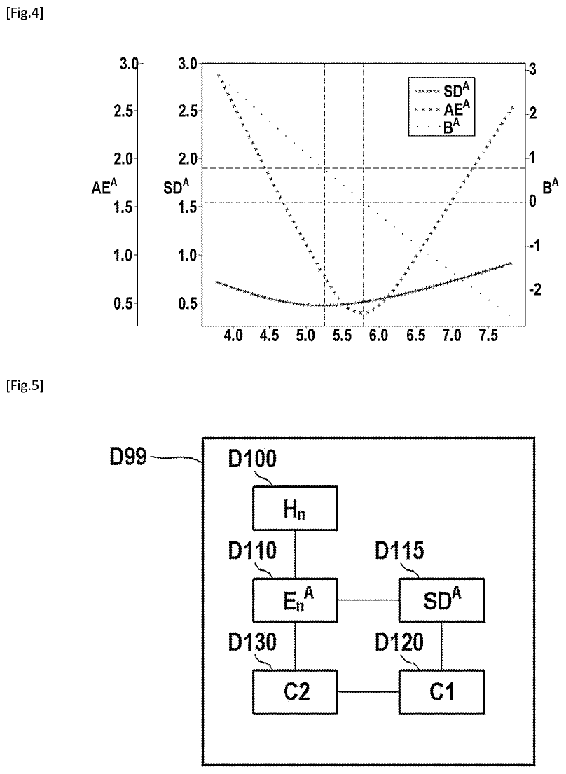

In reference to FIGS. 1 and 2, determining the adjustment values will be described, and in reference to FIGS. 3 and 4 the characterisation of a target system with such adjustment values will be described.

FIG. 1 illustrates the principal steps of a process for determining adjustment values intended to characterise a target system according to a particular embodiment of the invention.

These adjustment values are determined on the basis of a history of data relative to eye implant reference systems of the type of the target system. Each reference system comprises a reference eye and a reference implant.

In this way, in the mode described here, this determining process comprises a step E100 for obtaining a history of data H₁-H_{N}.

FIG. 2 illustrates a datum Hₙ in a particular embodiment of the invention.

In the embodiment described here, this datum Hₙ comprises one or more optical properties POₙ of a reference system SRₙ of the type of the target system, including at least one adjustable optical property PAₙ, the power Pₙ of the implant Iₙ of this reference system SRₙ comprising this implant Iₙ and a reference eye Oₙ, and a value CMₙ of an optical performance CP measured after the implant is placed and stabilised.

In a particular embodiment, the adjustable property PAₙ of a reference system SRₙ is the implant-cornea distance of this system which can take into account an angle between an axis of the reference eye and an axis of the reference implant.

The determining process comprises a calculation step E110, as a function of an adjustment variable A, for all data Hₙ of the history, of a predicted value CPₙ^{A} of the optical performance of the corresponding reference system SRₙ, and a prediction error Eₙ^{A} by creating the difference between this value CPₙ^{A} and the measured optical performance CMₙ.

In an embodiment in which the optical performance CP is a refractive correction, the determining process utilises the following formula EQ1:

$$\\ \begin{matrix}{E}_{n}^{{}_{} A} = {CP}_{n}^{{}_{} A} - {CM}_{n} = \frac{1}{\frac{1}{\frac{1}{\frac{{PA}_{n} + A}{{n}_{{a}_{n}}} + \frac{1}{\frac{{n}_{{v}_{n}}}{{AL}_{n} - ( {PA}_{n} + A )} - {P}_{n}}} - {V}_{{K}_{n}}} - {d}_{n}} - {CM}_{n} & ( EQ 1 )\end{matrix}$$

where ALₙ is the length of the reference eye (Oₙ), nᵥₙ is the refraction index of the ocular medium to the rear of the implant (the vitreous body), nₐₙ is the refraction index of the ocular medium in front of the implant (aqueous humour), V_{Kn} is the power of the cornea of the reference eye, and dₙ is the distance between the hypothetical spectacle lens and the cornea of the reference eye.

It should be noted that in the event where the calculated error Eₙ^{A} would be a difference between the calculated power Pₙ^{A} from the measured refractive correction CMₙ and the given power Pₙ of the reference implant Iₙ, the following formula could be used in place of the formula EQ1 to calculate this error Eₙ^{A}

$$\\ \begin{matrix}{E}_{n}^{{}_{} A} = {P}_{n}^{{}_{} A} - {P}_{n} = \frac{{n}_{{v}_{n}}}{{AL}_{n} - ( {PA}_{n} + A )} - \frac{{n}_{{a}_{n}}}{\frac{{n}_{{a}_{n}}}{\frac{1}{\frac{1}{{CM}_{n}} + {d}_{n}} + {V}_{{K}_{n}}} - ( {PA}_{n} + A )} - {P}_{n} & ( EQ 2 )\end{matrix}$$

In the calculation where the adjustable optical property PAₙ is an implant-cornea distance, the latter can especially be calculated by the formula EQ10 described hereinbelow.

In the embodiment described here, the determining process comprises a step E115 for calculation of dispersion SD^{A} of the errors Eₙ^{A} as a function of the adjustment variable A:

$$\\ \begin{matrix}{SD}^{{}_{} A} = \sqrt{{\sum }_{n = 1}^{N} \frac{{( {E}_{n}^{{}_{} A} - {B}^{{}_{} A} )}^{2}}{N}} & ( EQ 3 )\end{matrix}$$

where B^{A} is the bias, that is, the average for n of the errors Eₙ^{A}.

In the embodiment described here, the determining process comprises a step E120 for determining, from the formulae EQ1 and EQ2, the value of the variable A which minimises the dispersion SD^{A}.

An algorithm for minimising this dispersion can consist of calculating a plurality of values of A and choosing the value which corresponds to the smallest dispersion value SD^{A}.

Another example of an algorithm for determining the minimum of the dispersion SD^{A} is a gradient descent algorithm. There is a need for example for successive iterations, in each of which the derivative of dispersion dSD^{A}/dA is calculated, then a term proportional to this derivative is subtracted from the value of A, and then the value of the dispersion taken at this novel value of A is selected for the following iteration. This process can be terminated for example when the derivative of the dispersion has become very small.

The result of this is the adjustment value (C1) of optical property which minimises the dispersion SDA of the errors Eₙ^{A}:

$$\\ \begin{matrix}C 1 = {arg min}_{A} {SD}^{{}_{} A} & ( EQ 4 )\end{matrix}$$

During a step E130, the determining process produces the performance adjustment value C2, equal to the dispersion bias obtained at a minimum (or residual error), that is:

$$\\ \begin{matrix}C 2 = {B}^{{}_{} C 1} = \frac{{\sum }_{n = 1}^{N} {E}_{n}^{{}_{} C 1}}{N} & ( EQ 5 )\end{matrix}$$

A particular embodiment of obtaining these adjustment values C1 and C2 is illustrated in FIG. 3: C1 corresponds to the value of A which minimises the dispersion SD^{A} and C2 is the residual error obtained at this value of A. This figure also illustrates an adjustment value C0 which would correspond to the value of A which cancels the bias BA. It shows in this figure that this value C0 is far removed from the dispersion minimum C1.

FIG. 4 illustrates results obtained on a history of data corresponding to a set of eyes operated on with implants Tecnis PCB00 and for which calculation of the effective implant-cornea distance is made with the formula known as “Hoffer-Q formula” (there are many other formulae, for example the Olsen, Haigis, Holladay II formulae, the SRK T formula etc.). These results are the dispersion SD^{A} of errors, the bias BA and the average absolute error AE^{A}, all as a function of the adjustment variable A of the effective implant-cornea distance. In this example, the errors are differences between predicted values and measured values of the refractive correction of the implant.

It can be seen on the one hand that the minima of dispersion and of the average absolute error are quite distinct, and on the other hand that the minimum of the average absolute error is very close to zero of the bias.

FIG. 5 illustrates a device D99 for determining in keeping with a particular embodiment of the invention for determining adjustment values C1 and C2.

This device D99 comprises a module D100 for obtaining a history of data H₁-H_{N}.

This device D99 comprises a module D110 configured to obtain these data Hₙ and to calculate errors Eₙ^{A} from these data and as a function of an adjustment variable A.

This device D99 comprises a module D115 for calculation of the dispersion SD^{A} of these errors Eₙ^{A}.

The device D99 comprises a module D120 configured to utilise the dispersion SD^{A} provided by the module D115 and determine the value C1 of the variable A which minimises this dispersion SD^{A}.

The device D99 comprises a module 130 configured to utilise the value C1 provided by the module D120 and to obtain the average C2 of the errors Eₙ^{A} when the variable A is equal to the value C1.

For at least one type of eye implant system, executing the determining process produces an adjustment value C1 of optical property and a performance adjustment value C2. These adjustment values can be used to characterise a target eye implant system of this type.

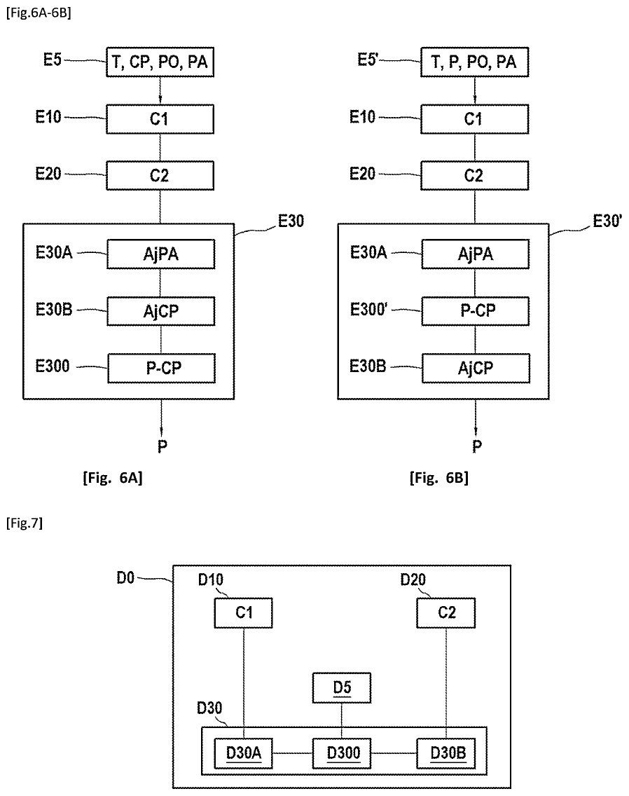

FIG. 6 illustrates two embodiments of a characterisation process in keeping with the invention (FIGS. 6A and 6B).

FIG. 6A illustrates a first embodiment of a characterisation process of a target system of a type T comprising a target implant and a target eye.

The process of FIG. 6A comprises a step E5 of type T of the target system for obtaining an optical performance CP of the target system, an optical property or properties PO of this target system including an adjustable optical property PA.

This process aims to produce the power P of the target implant which will produce this optical performance CP once the implant is placed in the target eye and stabilised.

In this embodiment, the optical performance CP is a refractive correction by the cornea of the target eye and the implant of the target system, which would be equivalent to the refractive correction contributed by a theoretical spectacle lens placed at a fixed distance d from this eye.

In this embodiment, the adjustable optical property PA is an implant-cornea distance.

During a step E10, an adjustment value C1 of optical property for eye implant systems of type T is obtained.

During a step E20, an adjustment value C2 of optical performance for eye implant systems of type T is obtained.

The adjustment value of optical property C1 is a value which has been determined to minimise dispersion of errors, each of the errors being a difference between an initially predicted value of the optical performance and a measured value of this optical performance for a reference system of type T of the target system.

The adjustment value of a performance C2 is a value which has been determined so as to cancel a bias of these errors when dispersion is minimal.

The characterisation process may produce these adjustment values C1 and C2 by any means. They are recorded for example in a table in association with a type of eye implant system.

In an example, these adjustment values C1 and C2 are determined by carrying out a determining process in keeping with the invention and whereof an example has been described in reference to FIG. 1.

In the embodiment described here, during a step E30, the process of FIG. 6A determines the power P from the optical performance CP, in three sub-steps.

A first sub-step E30A comprises adjustment of the value of the adjustable property PA by adding the optical performance CP and the adjustment value C1 of optical property.

A second sub-step E30B comprises adjustment of the value of the optical performance CP by subtracting the performance adjustment value C2 from the optical performance CP obtained at step E5.

A third sub-step E300 calculates the power P of the target implant from the optical property or properties PO, of the adjusted value of the optical performance CP and of the adjusted value of the adjustable optical property PA.

In the event where the optical performance CP is a refractive correction and where the adjustable optical property PA is an implant-cornea distance, this calculation can be based on a physical model of the eye which sets up a formula expressing the power P of the implant as a function of the refractive correction CP. An example of such a formula is:

$$\\ \begin{matrix}P = \frac{{n}_{v}}{AL - ( PA + C 1 )} - \frac{{n}_{a}}{\frac{{n}_{a}}{\frac{1}{\frac{1}{CP - C 2} + d} + {V}_{K}} - ( PA + C 1 )} & ( EQ 6 )\end{matrix}$$

where the length of the eye AL, the refraction index nᵥ of the ocular medium at the rear of the implant (the vitreous body), the refraction index nₐ of the ocular medium at the front of the implant (aqueous humour), the power V_{K} of the cornea, the distance d between a theoretical spectacle lens and the cornea, and the implant-cornea distance PA are optical properties PO of the target system.

This example shows that the equation EQ6 takes into account the adjusted values of the implant-cornea distance (PA+C1) and of the refractive correction (CP-C2).

If, according to another embodiment, the adjustment value C2 were intended to adjust the power P of the target implant, the formula EQ5 would be replaced by the formula:

$$\\ \begin{matrix}P = \frac{{n}_{v}}{AL - ( PA + C 1 )} - \frac{{n}_{a}}{\frac{{n}_{a}}{\frac{1}{\frac{1}{CP} + d} + {V}_{K}} - ( PA + C 1 )} - C 2 & ( EQ 7 )\end{matrix}$$

FIG. 6B illustrates a second embodiment of a characterisation process of a target system of type T comprising a target implant and a target eye.

The steps of FIG. 6B which bear the same reference as those of FIG. 6A are identical or similar to those of FIG. 6A.

The process of FIG. 6B comprises a step E5′ for obtaining, of type T of the target system, a power P of the target implant of the target system, an optical property or properties PO of this target system including an adjustable optical property PA.

This process aims to produce the optical performance CP of the target system which would be obtained once the target implant of power P is placed in the target eye and stabilised.

Step E5′ is followed by steps E10 and E20 already described for obtaining the two adjustment values C1 and C2.

In the embodiment described here, step E20 is followed by a step E30′ which determines the optical performance CP from the power P, in three sub-steps E30A, E300′ and E30B.

The first step E30A, identical or similar to that described in reference to FIG. 6A, adjusts the adjustable property PA obtained at step E5′.

In the embodiment described here, the second step E300′ calculates the optical performance CP from the power P, of the optical property or properties PO and of the value adjusted of the adjustable optical property PA.

The third step E30B, identical or similar to that described in reference to FIG. 6A, adjusts the optical performance CP obtained at step E300′.

In the calculation where the optical performance CP is a refractive correction and where the adjustable optical property PA is an implant-cornea distance, the inverse formula of that given written previously (EQ6) can be used to express the correction CP as a function of the power P, and by taking into account the two adjustment values C1,C2:

$$\\ \begin{matrix}CP = \frac{1}{\frac{1}{\frac{1}{\frac{PA + C 1}{{n}_{a}} + \frac{1}{\frac{{n}_{v}}{AL - ( PA + C 1 )} - P}} - {V}_{K}} - d} - C 2 & ( EQ 8 )\end{matrix}$$

If, according to another embodiment, the adjustment value C2 were intended to adjust the power P of the target implant, the formula EQ6 would be replaced by the formula:

$$\\ \begin{matrix}CP = \frac{1}{\frac{1}{\frac{1}{\frac{PA + C 1}{{n}_{a}} + \frac{1}{\frac{{n}_{v}}{AL - ( PA + C 1 )} - ( P - C 2 )}} - {V}_{K}} - d} & ( EQ 9 )\end{matrix}$$

In the embodiment of FIGS. 6A and 6B, the adjustable optical property PA was given as a process input (steps E0 or E0′). In another embodiment, the adjustable optical property PA, for example the effective implant-cornea distance is deducted from the other optical properties PO.

In the calculation where the adjustable optical property PA is an implant-cornea distance, the latter can be calculated by a wide range of formulae, generally deduced from statistical learning. Such a formula can for example result from linear regression, an example of which is:

$$\\ \begin{matrix}PA = {a}_{0} + {a}_{1} \times K + {a}_{2} \times AL + {a}_{3} \times WTW & ( EQ 10 )\end{matrix}$$

where a₀-a₃ are coefficients of regression, K, AL and WTW are properties of the eye in question (K: curvature of the cornea, AL: length of the eye, WTW: width of the cornea) and which constitute an example of optical properties (PO). It should be stressed that such a formula can be complicated by taking as input other optical properties and additional coefficients, and/or by using non-linear functions. It should also be noted that this formula can be secret.

FIG. 7 illustrates a device DO for characterisation of a target system in keeping with a particular embodiment of the invention.

This device D0 comprises a module D5 for obtaining:
a type T of the target system,
an optical performance CP of this target system or a power P of the target implant of this target system,
one or more optical properties PO of this target system including an adjustable optical property PA.

This device DO also comprises:
a module D10 for obtaining an adjustment value C1 of optical property,
a module D20 for obtaining an adjustment value C2 of optical performance,
a module D30 for determining a correspondence between a power of the target implant and an optical performance of the target system, itself comprising three sub-modules:
   (i) a sub-module for adjustment D30A of an adjustable optical property,
   (ii) a sub-module for adjustment D30B of an optical performance; et
   (iii) a sub-module D300 for calculation of a correspondence between the optical performance CP and the power P.

According to its configuration, this device DO is configured to carry out the characterisation process of the target system described previously in reference to FIG. 6A or 6B.

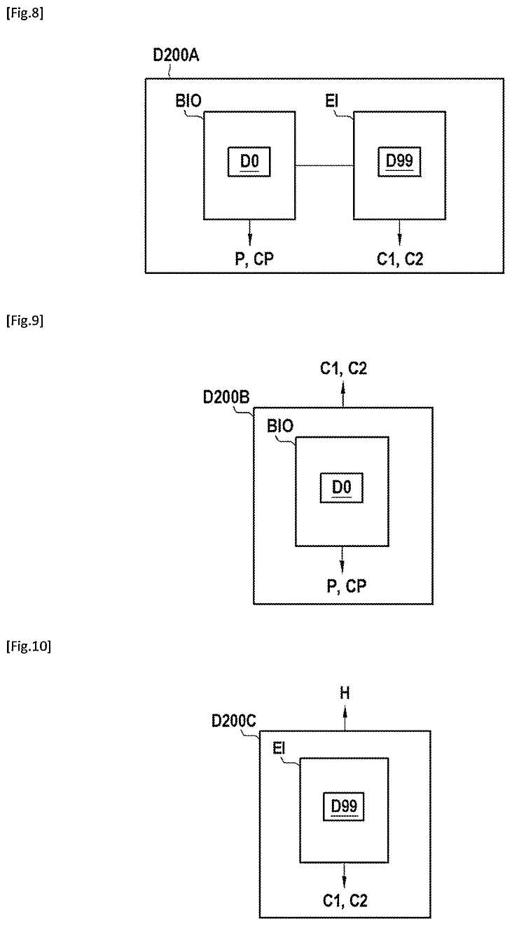

FIG. 8 illustrates an embodiment in which the prediction and determining processes such as described previously are put in place in a system D200A comprising a biometer BIO and a computer EI.

The biometer BIO comprises a device DO which executes the characterisation process of a target system by determining a power P of the target implant and an optical performance CP of the target system, for example its refractive correction.

For each target eye, the biometer BIO takes biometric measurements from which the optical properties PO of this eye are derived. The biometer also supplies an adjustable optical property PA, for example the predicted implant-cornea distance from the optical properties PO, by using the Hoffer-Q formula for example.

According to the prediction process described previously and put in place on the device D10, the optical performance CP, for example the refractive correction, is obtained from one or more optical properties PO including an adjustable optical property PA, for example the implant-cornea distance PA and adjustment values C1 and C2 adapted to the type T of the target implant and provided by the computer EI.

This computer EI comprises a device D99 putting in place a process for determining the adjustment values C1 and C2 such as described previously. The history H comprising the data Hₙ of eye implant reference systems SRₙ for determining the adjustment values C1 and C2 is recorded on a hard drive of the computer EI.

In a particular embodiment, this history is constructed with the biometer BIO of the same system. In this case, in addition to taking the biometric measurements on reference eyes, the biometer BIO can take measurements of refractive correction of eye implant reference systems after the placing and stabilising of the implant.

This specific case can for example correspond to the use, by a surgeon, of the system D200A both to characterise eye implant target systems and therefore prepare surgical operations for placing implants and to harvest pre- and postoperative data for determining novel adjustment values, with the aim of improving future characterisations of eye implant systems, thereby improving future operations of implant placement.

FIG. 9 illustrates an embodiment in which the characterisation process of a target implant as described previously is put in place by a system D200B comprising a biometer BIO. The biometer BIO comprises the device DO which executes this characterisation process. The execution of this process differs from its embodiment shown in FIG. 8 in that the adjustment values C1 and C2 are supplied by a third party to the system D200B.

FIG. 10 illustrates an embodiment in which the process for determining adjustment values C1 and C2 such as described previously is put in place in a system D200C comprising a computer EI. The computer EI comprises the device D99 which executes the process for determining adjustment values. The execution of this determining process differs from its embodiment shown in FIG. 8 in that the history H is supplied by a third party to the system D200C.

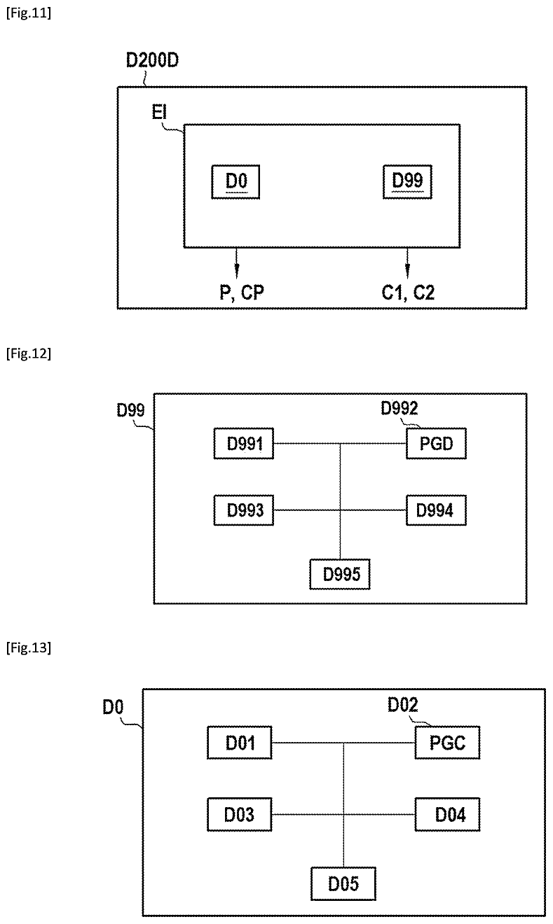

FIG. 11 illustrates an embodiment in which the prediction and determining processes such as described previously are put in place in a system D200D comprising a computer comprising both a characterisation device DO of a target system and the determining device D99. The difference with the embodiment shown in FIG. 8 is that the biometric measurements cannot be taken by this system and the optical properties PO are provided by a third party to the system D200D.

FIG. 12 illustrates the hardware architecture of a determining device D99 in keeping with a particular embodiment of the invention.

In the embodiment described here, the device for determining D99 has computer hardware architecture. It comprises in particular a processor D991, a read-only memory D992, a main memory D993, a rewritable non-volatile memory D994 and communication means D995.

The read-only memory D992 of the device D99 constitutes a storage medium in keeping with the invention, readable by the processor D991 and which stores a computer program PGD in keeping with the invention, this program comprising instructions for execution of the steps of a determining process according to the invention described previously in reference to FIG. 1 in an embodiment.

The computer program PGD defines functional modules of the determining device D99 shown in FIG. 5.

FIG. 13 illustrates the hardware architecture of a characterisation device DO in keeping with a particular embodiment of the invention.

In the embodiment described here, the characterisation device DO has computer hardware architecture. It comprises especially a processor D01, a read-only memory D02, a main memory D03, a rewritable non-volatile memory D04 and communication means D05.

The read-only memory D02 of the device DO constitutes a storage medium in keeping with the invention, readable by the processor D01 and on which a computer program PGC is recorded in keeping with the invention, this program comprising instructions for executing the steps of a characterisation process according to the invention described earlier in reference to FIGS. 6A and 6B in two embodiments.

The computer program PGC defines functional modules of the characterisation device D0 illustrated in FIG. 7.

1. A process executed by computer for characterising a target system comprising a target implant and a target eye, said target system being characterised by an optical performance and a power of said target implant, said process comprising:
obtaining an adjustment value of optical property intended to adjust at least one adjustable optical property of said target system;
obtaining an adjustment value of optical performance of said target system;
determining a correspondence between said power and the optical performance from at least one optical property of the target system including at least one adjustable property, of said adjustment value of optical property and of said performance adjustment value, at least one of said optical properties being obtained from measurements of the target eye;
wherein said determining of a correspondence between said power and the optical performance comprises:
determining of said optical performance as a function of said power; or
determining of said power as a function of said optical performance, and wherein the determining comprises:
(i) adjustment of a value of said adjustable property with said adjustment value of optical property, and
(ii) adjustment of a value of said optical performance or of said power with said performance adjustment value,
said adjustment value of optical property having been determined for minimising dispersion of errors, each of said errors being:
(i) a difference between an initially predicted value, from a given value of said power, of said optical performance and a measured value of said optical performance, or
(ii) a difference between an initially predicted value, from the measured value of said optical performance, of said power and the given value of said power,
for a reference system of a type of the actual target system and comprising a reference eye and a reference implant;
said performance adjustment value having been determined so as to cancel a bias of said errors obtained when said dispersion is minimal.

2. The characterisation process according to claim 1 wherein said at least one adjustable optical property is a distance between the target implant and one of the elements of the target eye.

3. The characterisation process according to claim 1, wherein at least one of said optical properties is obtained from biometric measurements of the target eye.

4. The characterisation process according to claim 1, wherein at least one of said adjustable properties is calculated from at least one of said optical properties.

5. The characterisation process according to claim 4 according to which the calculation of at least one of said adjustable properties is determined from a statistical regression or machine learning technique.

6. A process executed by computer for determining adjustment values intended to characterise a target system comprising a target implant and a target eye, said target system being characterised by an optical performance and a power of said target implant, said process comprising:
obtaining a history comprising, for at least one reference system of a type of said target system, a datum comprising:
(i) a measured value of its optical performance,
(ii) at least one of its optical properties including at least one adjustable property; and
(iii) a given value of the power of its reference implant;
calculation as a function of the same adjustment variable for all reference systems:
(i) for said at least one reference system, and by using its actual datum, of an error being:
(i-a) a difference between an initially predicted value and said measured value of its optical performance, or
(i-b) a difference between an initially predicted value and said given value of the power of its implant; and
(ii) of dispersion of said errors obtained for a set of said reference systems;
determining an adjustment value of optical property intended to adjust at least one adjustable property of said target system and corresponding to the value of said adjustment variable which minimises said dispersion, and
determining a performance adjustment value, equal to an average of said errors when said adjustment variable is equal to said adjustment value of optical property, and intended to adjust said optical performance or said power of the target system.

7. The process for determining according to claim 6 wherein the value of said adjustment variable which minimises said dispersion is determined by a gradient descent algorithm of said dispersion as per said variable, or by an algorithm consisting of calculating said dispersion for a plurality of values of said adjustment variable and selecting the value which minimises said dispersion.

8. The process for determining according to claim 6, wherein said at least one adjustable optical property of each reference system corresponds to a distance between the reference implant and one of the elements of the reference eye.

9. The process for determining according to claim 6, wherein at least one optical properties of at least one reference system is obtained from biometric measurements of the eye of this reference system.

10. The process for determining according to claim 6, wherein at least one of the adjustable properties of at least one reference system is calculated from at least one of said optical properties of this the at least one reference system.

11. The process for determining according to claim 10 according to which the calculation of at least one of said adjustable properties of at least one reference system is determined from a regression or machine learning technique.

12. The characterisation process according to claim 1, wherein the determining of said adjustment value of optical property and of said performance adjustment value is carried out by operations comprising:
obtaining a history comprising, for at least one reference system of a type of said target system, a datum comprising:
(i) a measured value of its optical performance,
(ii) at least one of its optical properties including at least one adjustable property, and
(iii) a given value of the power of its reference implant;
calculation as a function of the same adjustment variable for all reference systems:
(i) for said at least one reference system, and by using its actual datum, of an error being:
(i-a) a difference between an initially predicted value and said measured value of its optical performance, or
(i-b) a difference between an initially predicted value and said given value of the power of its implant; and
(ii) of dispersion of said errors obtained for a set of said reference systems;
determining an adjustment value of optical property intended to adjust at least one adjustable property of said target system and corresponding to the value of said adjustment variable which minimises said dispersion, and
determining a performance adjustment value, equal to an average of said errors when said adjustment variable is equal to said adjustment value of optical property, and intended to adjust said optical performance or said power of the target system.

13. The process according to claim 1, wherein said optical performance corresponds to at least one of a property from among:
a refraction,
a refractive correction,
a position for focusing refracted light rays, and
a correction of contrast.

14. The process according to claim 1, wherein two actual eye implant systems are of the same type if they have in common at least:
a characteristic of the implants of said systems;
a characteristic of the eyes of said systems; or
a characteristic of the interactions between the eye and the implant of said systems.

15. The process according to claim 1, wherein a single adjustment value of optical property and a single performance adjustment value are used.

16. A device for characterisation of a target system comprising a target implant and a target eye, said target system being characterised by an optical performance and a power of said target implant, said device comprising:
a module for obtaining an adjustment value of optical property intended to adjust at least one adjustable optical property of said target system,
a module for obtaining an adjustment value of optical performance of said target system;
a module for determining a correspondence between said power and the optical performance from at least one optical property of the target system including at least one adjustable property, of said adjustment value of optical property and of said performance adjustment value, at least one of said optical properties being obtained from measurements of the target eye;
wherein the determining of a correspondence between said power and the optical performance comprises:
determining of said optical performance as a function of said power; or
determining of said power as a function of said optical performance, and wherein the determining comprises:
(i) adjustment of a value of said adjustable property with said adjustment value of optical property, and
(ii) adjustment of a value of said optical performance or of said power with said performance adjustment value,
said adjustment value of optical property having been determined for minimising dispersion of errors, each of said errors being:
(i) a difference between an initially predicted value, from a given value of said power, of said optical performance and a measured value of said optical performance, or
(ii) a difference between an initially predicted value, from the measured value of said optical performance, of said power and the given value of said power,
for a reference system of a type of the actual target system and comprising a reference eye and a reference implant;
said performance adjustment value having been determined so as to cancel a bias of said errors obtained when said dispersion is minimal.

17. A device for determining adjustment values intended to characterise a target system comprising a target implant and a target eye, said target system being characterised by an optical performance and a power of said target implant, said device comprising:
a module for obtaining a history comprising, for at least one reference system of a type of said target system, a datum comprising:
(i) a measured value of its optical performance,
(ii) at least one of its optical properties including at least one adjustable property; and
(iii) a given value of the power of its reference implant;
a calculation module as a function of the same adjustment variable for all the reference systems:
(i) for said at least one reference system, and by using its actual datum, of an error being:
(i-a) a difference between an initially predicted value and said measured value of its optical performance, or
(i-b) a difference between an initially predicted value and said given value of the power of its implant;
(ii) of dispersion of said errors obtained for a set of said reference systems;
a module for determining an adjustment value of optical property intended to adjust at least one adjustable property of said target system and corresponding to the value of said adjustment variable which minimises said dispersion, and
a module for determining a performance adjustment value, equal to an average of said errors when said adjustment variable is equal to said adjustment value of optical property, and intended to adjust said optical performance or said power of the target system.

18. A system comprising a biometer and/or computer equipment, each comprising at least one device according to claim 16.

19. (canceled)

20. A nontransitory recording medium readable by computer equipment and/or a biometer, comprising instructions which, when executed by the computer equipment or the biometer, perform the process according to claim 1.
